(19) United States
(12) Patent Application Publication
(10) Pub. No.: US 2004/0259256 A1
(43) Pub. Date: Dec. 23, 2004

(54) Methods of unnatural amino acid incorporation in mammalian cells

(75) Inventors: Sarah Monahan, Pasadena, CA (US); Dennis A. Dougherty, South Pasadena, CA (US); Henry A. Lester, Pasadena, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Neurion Pharmaceuticals

(21) Appl. No.: 10/805,169

(22) Filed: Mar. 19, 2004

Related U.S. Application Data
(60) Provisional application No. 60/456,234, filed on Mar. 21, 2003.

(51) Int. Cl. C12P 21/06; C12N 15/87

(52) U.S. Cl. 435/459

(57) ABSTRACT

The present invention discloses a general method for the efficient delivery of suppressor tRNA and mRNA or DNA encoding a protein of interest to mammalian cells. The method of electroporation described herein is useful for expanding nonsense suppression methodology to mammalian cell expression systems for the incorporation of unnatural amino acids.

### CROSS-REFERENCES TO RELATED APPLICATIONS

This application claims benefit of priority from U.S. Provisional Patent Application 60/456,234, filed Mar. 21, 2003, which is hereby incorporated in its entirety as if fully set forth.

### FIELD OF THE INVENTION

The present invention generally relates to methods of unnatural amino acid incorporation into proteins expressed in mammalian cells. The present invention more specifically relates to the use of electroporation as an effective and general method for the delivery of mRNA, DNA, and tRNA into a variety of adherent mammalian cell types. The present invention also relates to methods of modeling structure-function relationships of receptor proteins, drug discovery and design, and medical research and diagnostic imaging using mammalian cell expression systems.

### BACKGROUND OF THE INVENTION

Unnatural amino acid incorporation into ion channels has proven to be an invaluable tool for structure-function studies (Dougherty (2000) Curr. Opin. Chem. Biol. 4: 645-652). Using the in vivo nonsense suppression methodology (Nowak et al. (1998) Methods in Enzymol. 293:515), extensive studies have been done on a variety of ion channels including the nicotinic ACh receptor (nAChR), 5-HT3_{A} receptor, and the Shaker and Kir2.1 potassium channels. To date such studies have been limited to the Xenopus oocyte heterologous expression system. While a great deal of information on ligand binding and ion channel gating mechanisms has been obtained from such studies, there are clear benefits in the art to expanding the technology to a mammalian cell expression system. This would provide a more relevant environment for many proteins of mammalian origin, and would allow for studies of more complex signaling cascades.

The present invention provides for unnatural amino acid incorporation in mammalian cells. The present invention provides a general method to deliver mRNA encoding a protein of interest, an amber suppressor tRNA, and a reporter gene to mammalian cells. Co-electroporation of a human amber suppressor tRNA (RajBhandary et al. (2001) Proc. Nati. Acad. Sci. USA 98(25): 143 10-14315) with the DNA or mRNA corresponding to the protein of interest into adherent cells leads to highly efficient delivery of these components and highly efficient nonsense suppression, as demonstrated by expression of both green fluorescent protein (EGFP) and nAChR in CHO-Ki and IIEK cells as well as in cultured hippocampal neurons. The use of the amber suppressor tRNA THG73 (Nowak et al. (1998) Methods in Enzymol. 293:515), chemically acylated with natural or unnatural amino acids, in the method of the present invention leads to efficient delivery of the amino acid site—specifically into nAChR expressed in CHO cells. The present invention demonstrates that electroporation is an effective and general method to deliver not only mRNA and DNA, but also tRNA into a variety of adherent mammalian cell types, and that in vitro transcribed suppressor tRNA is fully functional in these cells. The present invention thus expands the nonsense suppression methodology to unnatural amino acid incorporation for mammalian cell expression systems.

Citation of the above documents is not intended as an admission that any of the foregoing is pertinent prior art. All statements as to the date or representation as to the contents of these documents is based on the information available to the applicant and does not constitute any admission as to the correctness of the dates or contents of these documents.

### SUMMARY OF THE INVENTION

A general method to deliver nucleic acid encoding a protein of interest, tRNA, and a reporter gene to mammalian cells is disclosed herein. More specifically, the method disclosed herein involves co-electroporation of a tRNA transcript with the DNA or mRNA corresponding to the protein of interest into adherent cells leading to highly efficient delivery of these components and highly efficient nonsense suppression. Furthermore, the method disclosed herein provides for unnatural amino acid incorporation in mammalian cells, thereby expanding nonsense suppression methodology to applications such as studies of cell signaling cascades, probing structure-function relationships of receptor proteins, drug discovery and design, and medical research and diagnostic imaging.

A first aspect of the invention is to provide a method for the delivery of tRNA along with nucleic acid corresponding to a protein of interest to mammalian cells comprising transfecting cultured mammalian cells with the tRNA and the nucleic acid corresponding to the protein of interest. Compositions comprising the tRNA and corresponding nucleic acid are also provided for transfection into the cells.

Another aspect of the invention is to provide a method for the incorporation of unnatural amino acids into mammalian cells, comprising a) incorporating a nonsense codon into the nucleic acid corresponding to a protein of interest, b) producing a tRNA transcript that contains the corresponding anticodon to the nonsense codon, c) chemically aminoacylating the tRNA transcript with the desired unnatural amino acid, and d) transfecting cultured mammalian cells with the aminoacyl tRNA and the nucleic acid corresponding to the protein of interest.

It is yet another aspect of the invention to provide a method for the incorporation of imaging reagents into mammalian cells, comprising a) incorporating a nonsense codon into the nucleic acid corresponding to a protein of interest, b) producing a tRNA transcript that contains the corresponding anticodon to the nonsense codon, c) chemically aminoacylating the tRNA transcript with the desired imaging reagent, and d) transfecting cultured mammalian cells with the aminoacyl tRNA and the nucleic acid corresponding to the protein of interest.

It is a further aspect of the invention to provide an in vitro tRNA transcript comprising a chemically aminoacylated suppressor tRNA that is used for the incorporation of unnatural amino acids into mammalian cells.

It is another aspect of the invention to provide an in vitro tRNA transcript comprising a chemically aminoacylated suppressor tRNA that is used for the incorporation of imaging reagents into mammalian cells.

Compositions comprising such tRNAs, optionally with a DNA or RNA as described herein, in a medium for transfer to cells are also provided.

It is yet another aspect of the invention to provide a method of determining the specific physiological effect of a compound on the activity of its receptor comprising developing a receptophore model, wherein said model allows for generation of compounds that can selectively modulate a receptor subtype in a specific receptor conformation to achieve a desired physiological activity; using nonsense suppression methodology to determine details of the nature and location of receptor binding of said compounds; and using said receptophore model to predict which compound could achieve said physiological activity on the target receptor by evaluating how, where, and in what receptor conformation state said compound binds to the receptor.

Another aspect of the invention is to provide a method of determining the nature of a compound's interaction with a receptor comprising a) incorporating unnatural amino acids into binding and regulatory sites of the receptor, resulting in an altered receptor, b) measuring the compound's ability to bind to the altered receptor, and c) comparing the results of step (b) to the same compound's ability to bind to an unaltered receptor.

It is a further aspect of the invention to provide a method of identifying key interactions that would lead to discovery of new compounds comprising a) determining the nature of the compound's interaction with the receptor, b) analyzing how and where the compound interacts with the receptor, and c) based on the analysis in step (b), chemically modifying the compound to achieve desired ligand activity.

It is yet a further aspect of the invention to provide a screening methodology comprising a membrane protein receptor which has been modified to replace native amino acids with unnatural amino acids, wherein the receptor is expressed in vivo by mammalian cells.

### BRIEF DESCRIPTION OF THE DRAWINGS

FIG. 1. Scheme showing incorporation of unnatural amino acids into the receptor in a Xenopus oocyte expression system.

FIG. 2. Fluorescence images of CHO-K1 cells electroporated with either A) HSAS and mutant S29TAG pEGFP-F DNA or B) wild type EGFP-F.

FIG. 3. Fluorescence images of rat hippocampal neurons electroporated with A) HSAS and Ser29TAG pEGFP-F DNA, B) wt EGFP-F DNA, or C) Ser29TAG pEGFP-F DNA in the absence of HSAS tRNA (read-through).

FIG. 4. (A) Electrophysiological traces showing responses to ACh application, of CHO cells transfected with the wild type nAChR subunits; with the β Leu9′Ser mutant and the remaining wild type subunits; with mutant β Leu9′TAG mRNA, the remaining wild type subunits and HSAS tRNA; and with mutant Leu9′TAG, in the absence of HSAS tRNA (read-through). (B) ACh dose response curves for wt nAChR, β Leu9′Ser nAChR, and HSAS suppressed β Leu9′TAG nAChR.

FIG. 5. (A) Electrophysiological traces showing wild type recovery of the nAChR by suppression of α149TAG mRNA with THG73 tRNA aminoacylated with tryptophan (THG-W); and of the β9′Ser mutant nAChR channel by suppression of α149TAG mRNA with THG-W. (B) Electrophysiological traces showing ACh response from a cell expressing the unnatural amino acid 5,7-difluorotryptophan (THG-F₂W at α149 of the nAChR, and no ACh response from cells transfected with only mRNA, with or without uncharged tRNA. (C) ACh dose response curves for THG-W suppressed α149TAG/β9′Ser nAChR and THG- F₂W suppressed α149TAG/β9′Ser nAChR.

FIG. 6. Plot of log[EC₅₀/EC_{50(wt)}] vs. cation-π binding ability at α-Trp 149 of the nicotinic acetyicholine receptor for the wild type trp and the fluorinated trp derivatives 5-F-Trp, 5,7-F₂-Trp, 5,6,7-F₃-Trp, and 4,5,6,7-F₄-Trp.

### DETAILED DESCRIPTION OF THE INVENTION

The present invention encompasses a general electroporation method to transfect mammalian cells with suppressor tRNA and mRNA and DNA simultaneously. In vitro transcribed human amber suppressor tRNA (HSAS) is readily aminoacylated by CHO-K1 cells, HEK cells, and hippocampal neurons, and this tRNA is efficiently used by the translational machinery of these cells. Efficient expression of EGFP and nAChR in mammalian cells is encompassed using the suppression assay of the present invention. This illustrates that HSAS is recognized by endogenous serine synthetases, and delivers serine during translation.

The methods of the present invention further provides for the insertion of amino acid analogues into mammalian cells. The site-specific incorporation of unnatural amino acids using nonsense suppression methodology has already been shown in membrane receptor proteins expressed by Xenopus oocytes. Nowak et al. (1995) Science 268: 439-442; Dougherty (2000) Curr. Opin. Chem. Biol. 4: 645-652. The present invention expands this technique by demonstrating site-specific incorporation of unnatural amino acids into membrane receptors or ion channels in mammalian cells using chemically aminoacylated tRNA. The aminoacylated amber suppressor tRNA THG73 is shown to site-specifically deliver the appended amino acid to nAChR in CHO cells. This is shown using a nAChR suppression assay for THG73 aminoacylated with either wild-type Trp or a difluorotryptophan.

The development of a method for site-specific incorporation of unnatural amino acids into proteins in vivo using mammalian cell systems greatly expands the scope and utility of unnatural amino acid mutagenesis. Liu and Schultz (1999) Proc. Nail. Acad. Sci. USA 96:4780-4785; Kowal et al. (2001) Proc. Nail. Acad. Sci. USA 98: 2268-2273. In particular, such in vivo systems will find utility in in vivo structure-function studies, including studies of protein-protein interactions and protein localizations, that will be used to develop receptophore models for drug design and discovery applications. Such in vivo systems will also find utility in cell-based imaging assay applications, such as in cancer research and diagnostics, through the incorporation of imaging reagents into proteins in vivo.

The site-specific incorporation of a specific unnatural amino acid into proteins in mammalian cells was demonstrated by Yokoyama and coworkers. Sakamoto et al. (2002) Nucleic Acids Res. 30(21): 4692-469). A suppressor tRNA(Tyr) and mutant tyrosyl-tRNA synthetase pair was used to incorporate a mutant tyrosine into mammalian cells. With this approach, specific tRNA/aminoacyl tRNA synthetase pairs are required for the insertion of each unnatural amino acid. The amino acids are supplied in the cell growth medium and incorporated at amber stop positions by the variant enzymes and the cognate suppressor tRNA. The method of the present invention does not utilize engineered tRNA synthetases for unnatural amino acid incorporation. Instead, the suppressor tRNA of the present invention is supplied to the cells in aminoacylated form, thus bypassing the need to add the specific variant synthetase and providing a broader range of applicability to in vivo unnatural amino acid incorporation.

Nonsense suppression of EGFP with aminoacyl-tRNA has also been shown by Vogel and coworkers. Ilegems et al. (2002) Nucleic Acids Res. 30: e128. CHO cells were microinjected with in vitro transcribed E. coli amber suppressor tRNA chemically aminoacylated with leucine, along with the Leu64TAG mutant EGFP mRNA reporter gene, leading to the recovery of wild type EGFP expression. The method of the present invention uses electroporation for gene delivery, which is more general, less tedious, and less destructive than single-cell gene transfer methods such as microinjection.

The human serine amber suppressor tRNA (HSAS) of the present invention was shown to successfully act as a suppressor of amber codons when imported into mammalian COS1 cells. RajBhandary et al. (2001) Proc. Nati. Acad. Sci. USA 98(25): 14310-143 15. The suppressor tRNA was purified from E. coli, chemically acylated, and inserted using the transfection reagent Effectene into COS 1 cells. Transfected cells were harvested and an in vitro CAT assay was used to characterize the import and function of the aminoacyl tRNA. For studies at the single cell level, such as those contemplated by the present invention, Effectene transfection appears to be unsuccessful at tRNA delivery.

The method of the present invention is the first to use electroporation as an efficient means of delivering the suppressor tRNA and DNA to mammalian cells. The advantages of electroporation over other transfection techniques, such as transfection reagents, microinjection, and biolistics, include higher transfection efficiency (up to 90%) and less cell death (Tereul and Meyer, Biophys. J. 1997, 73: 1785-1796; Tereul et al., J. Neurosci Meth.1999, 93: 37-48). The microelectroporator apparatus of the present invention requires a small volume of aminoacyl-tRNA, and protein expression can be seen as soon as two hours after transfection without compromise of cell health. Further, the present invention is the first to demonstrate incorporation of an unnatural amino acid into a receptor expressed in a mammalian cell. In vivo assays for tRNA delivery and function at the single cell level are shown with transfected mammalian cells using the electroporation method of the present invention.

The invention also provides for compositions comprising tRNAs and nucleic acids as described herein in a form suitable for electroporation. Such compositions may comprise a tRNA and DNA or RNA in any solution suitable for electroporation. Non-limiting examples of such a solution include CO₂ independent media suitable for mammalian cells, buffered solutions suitable for cells and electroporation, and buffered solutions with low conductivity for electroporation. The compositions are preferably sterile and are free of of mycoplasma, endotoxins and pyrogens. The invention also provides for co-precipitates of a tRNA and nucleic acid molecule, both as described herein. Such co-precipitates are preferably those which may be reconstituted for use in electroporation.

As used herein, a “receptophore” is the ensemble of steric and electronic features of a biological target that is necessary to ensure optimal supramolecular interactions with a specific ligand and to trigger or block the biological function of the target.

As used herein, an “unnatural amino acid” is any amino acid other than one of the 20 recognized natural amino acids as provided in Creighton, Proteins (W.H. Freeman and Co. 1984) pp2-53.

Protein Expression in Mammalian Cells from Nonsense Suppression

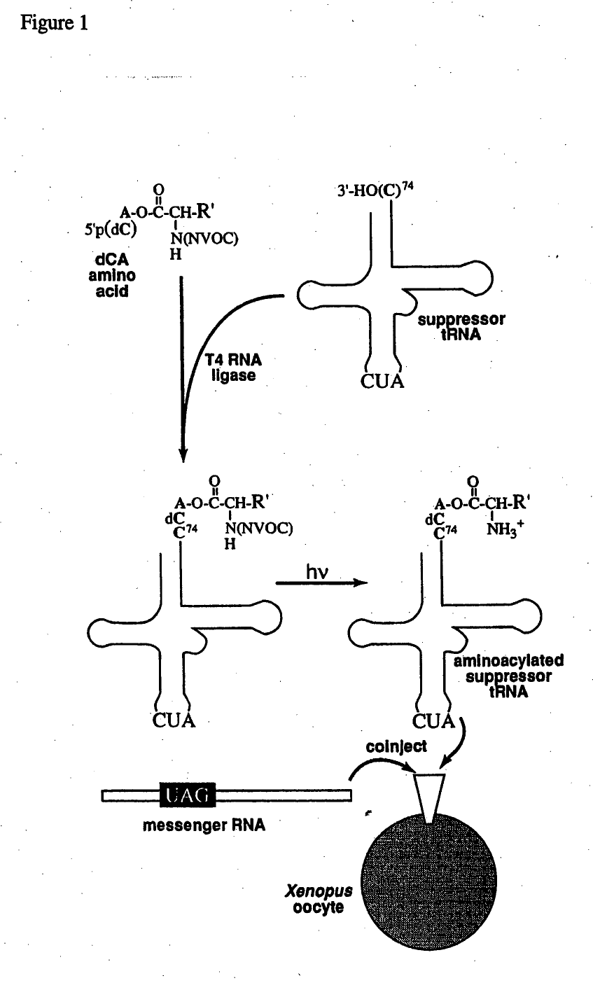

Amino acids are incorporated into specific sites on the protein through the use of nonsense codon suppression. Noren et al. (1989) Science 244:182; Nowak et al. (1998) Methods in Enzymol. 293:5 15. See FIG. 1. In the nonsense suppression method, two RNA species are prepared using standard techniques for example, such as in vitro synthesis from linearized plasmids. The first RNA species is an mRNA encoding the protein of interest but engineered to contain an amber stop codon (UAG) at the position where amino acid incorporation is desired. The second is a suppressor tRNA that contains the corresponding anticodon (CUA) and that is compatible with the expression system employed, such as, for example, Tetrahymena thermophila tRNA^{Gln}G73 (THG73). For the incorporation of unnatural amino acids, the tRNA is chemically acylated at the 3′ end with the desired unnatural amino acid using techniques known in the art such as that described in Kearney et al. (1996), Mol. Pharmacol., 50: 1401-1412.

Synthesis of the unnatural amino acids depends on the desired structure. The unnatural amino acid may be prepared, for example, by modification of natural amino acids. Also, many unnatural amino acids are commercially available. The NRC Biotechnology Research Institute Peptide/Protein Chemistry Group maintains an excellent listing of commercially available amino acids at aminoacid.bri.nrc.ca.

Examples of preferred unnatural amino acids for incorporation into mammalian cells using the methods of the present invention include, but are not limited to, those represented by the following Formula (I):

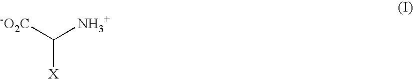

where X is selected from the group consisting of:

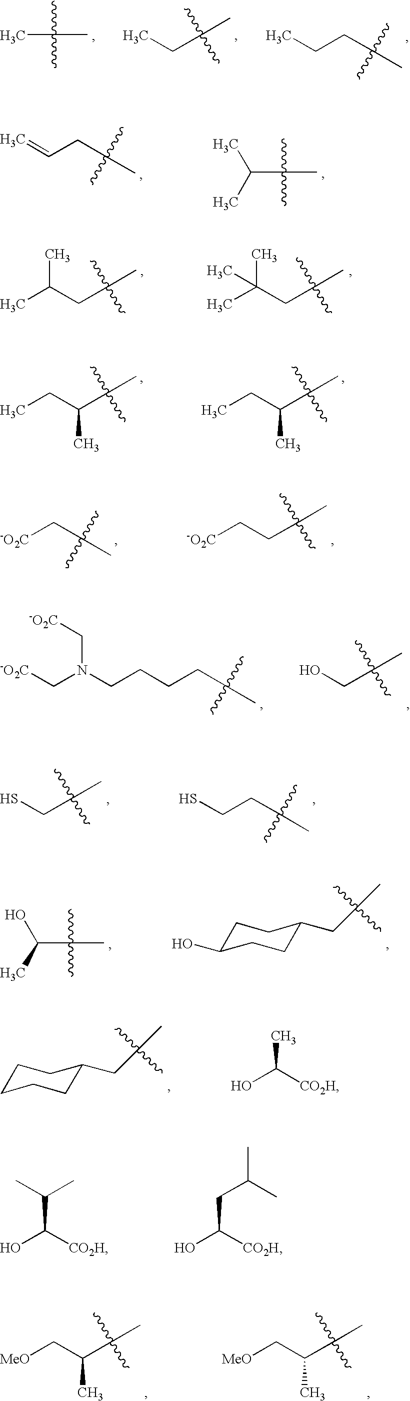

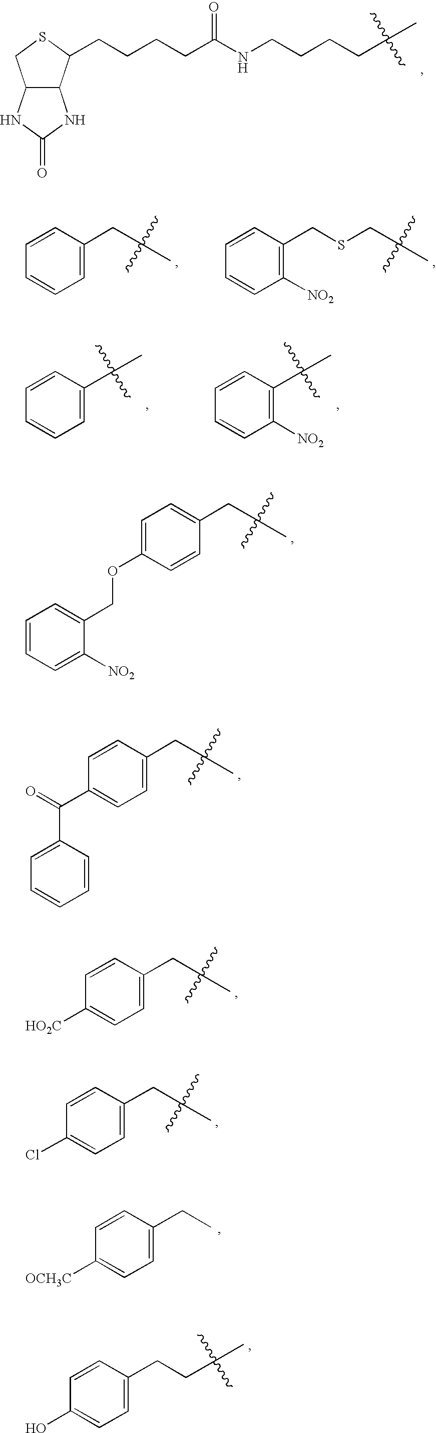

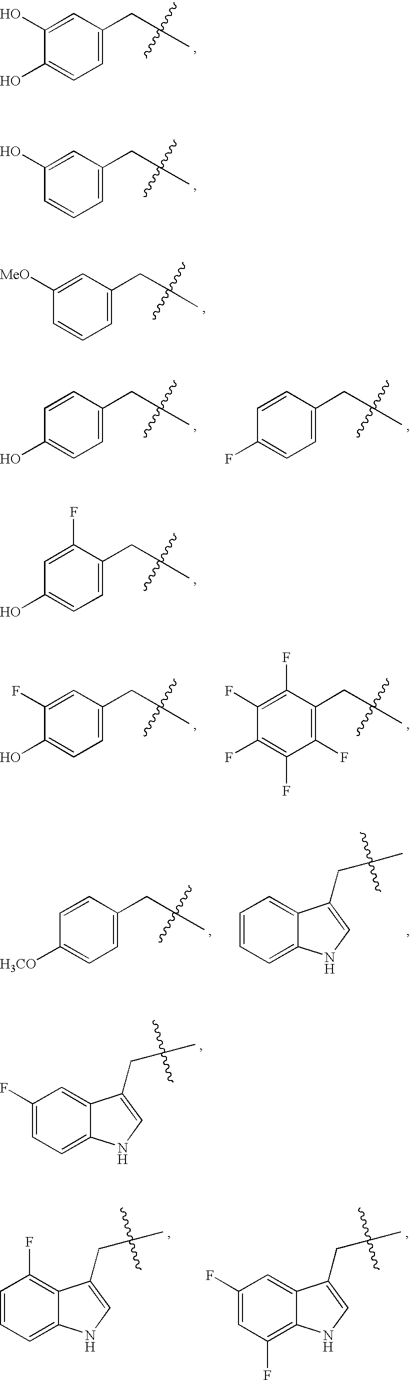

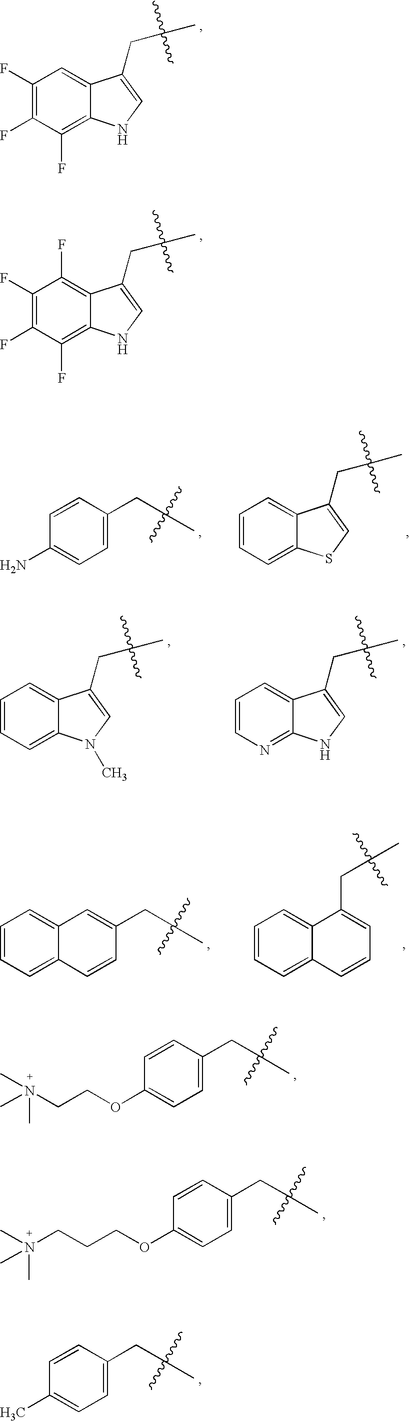

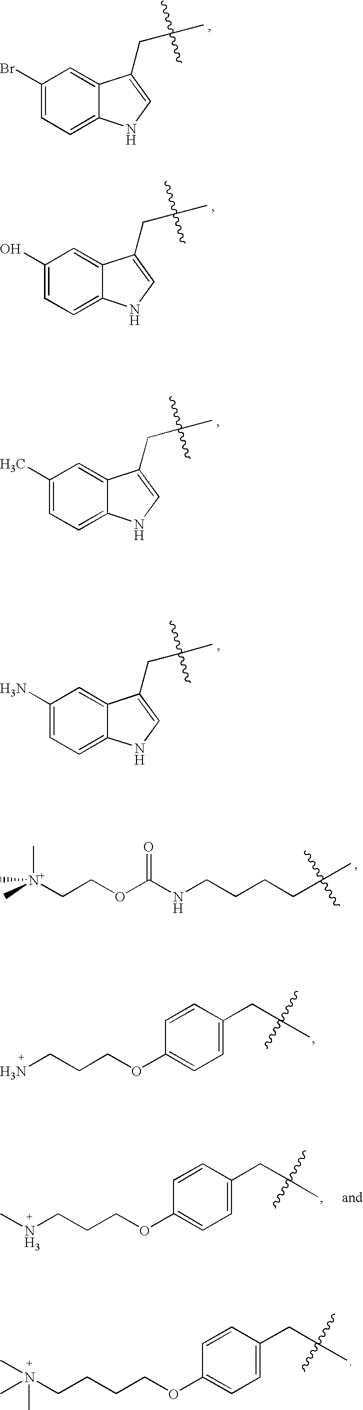

In another preferred embodiment, examples of unnatural amino acids for incorporation into mammalian cells also include, but are not limited to, those represented by the following Formula (II):

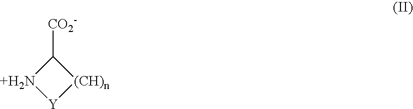

wherein: Y is CH₂, (CH)ₙ, N, O, or S, and n is 1 or 2. Examples of such compounds include, but are not limited to, the following compounds:

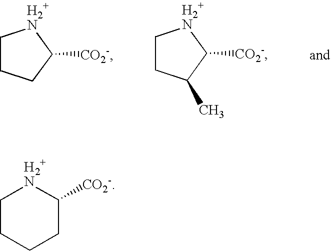

Note also that racemic amino acids can be used because only L-amino acids, and not D-amino acids, are incorporated. Cornish, et al. (1995) Angew. Chem.Int. Ed. Engl. 34: 621-633.

In a preferred embodiment, after synthesis of the relevant mRNA and suppressor tRNA, the species are co-electroporated into mammalian cells using standard procedures known in the art, such as electroporation (Teruel et al.). The electroporator is designed to transfect a small section (˜1 cm²) of cells in a 35 mm dish, and therefore may be practiced with only small volumes of the transfection solution, typically 5 μl with varying concentrations of DNA and RNA. During translation of the mRNA, the ribosome incorporates the amino acid encoded by the suppressor tRNA into the nascent peptide at the position of the engineered stop codon, and the corresponding protein is expressed by the cells.

After transfection, expression of the protein of interest may be measured with patch clamp electrophysiology using standard procedures known in the art. If expression of the protein of interest leads to a distinct phenotype, the skill artisan may design a suppression assay by measuring phenotype expression. The protein of interest can be a reporter protein such as, for example, luciferase, green fluorescent protein (EGFP), or other known fluorescent or other indicator proteins. In a preferred embodiment, nonsense suppression of an amber stop codon engineered into green fluorescent protein (EGFP) leads to EGFP expression, and is measurable through fluorescence imaging techniques.

Further, receptors may be used in the inventive method for study and discovery of novel agonist/antagonists. Such receptors include, but are not limited to, all ligand-gated ion channels. In a preferred embodiment, agonist dose response curves are used to compare expression of wild-type nicotinic acetylcholine receptor (nAChR) versus receptors generated from both mutant and tRNA-suppressed mutant mRNA. Although different proteins may be expressed in mammalian cells using the nonsense suppression method of the present invention, the invention is not limited by the protein of interest or by the method of detecting its expression.

Expression of the protein of interest occurs in the inventive method in mammalian cells. Any mammalian cell line may be used for the inventive method, including but not limited to CHO and/or HEK cells. A preferred embodiment for electroporation and subsequent expression of the protein of interest is the CHO-K1 cell line. Although many different mammalian cells and cell lines may be used for expression of proteins of interest using the nonsense suppression method, the choice of mammalian cells or cell line is not intended to limit the invention.

In vivo Nonsense Suppression for Cell-Based Research and Diagnostic Applications

The method of the present invention will be used in cell-based assay applications, such as ones used in cancer research and diagnostics, through the incorporation of imaging reagents into proteins in vivo. The site-specific labeling of recombinant proteins provides an extremely useful tool for the direct visualization of cellular events that is not possible with the use of bulky protein labels known in the art. In one preferred embodiment, fluorescent amino acids will be incorporated site-specifically into the protein of interest using the method of the present invention. A fluorescence-based assay will be used to determine the efficiency of probe incorporation. A number of different imaging reagents are known in the art and the invention is not limited to any single reagent or diagnostic application.

Generation of Receptophore Model for Drug Discovery and Design

Integral membrane protein receptors contain many transmembrane segments. For this and other reasons, there are very few examples of successful attempts to generate enough pure, properly folded, and functional proteins for high-resolution structural methods such as x-ray crystallography or NMR spectroscopy. The methods included herein describe the construction of a receptophore model using unnatural amino acid substitutions as an alternative, and further describe the use of the receptophore model to identify and refine potential ligands.

An accurate receptophore model is built through identification of amino acids involved in the ligand binding site and the probing of the molecular forces involved. First, unnatural amino acids are incorporated into target receptors using nonsense suppression methodology. Altered receptors are expressed heterologously by mammalian cell membranes. Compounds found to modulate a receptor subtype in a specific receptor conformation are screened for binding efficacy to the altered receptor. Electrophysiological or biochemical assays are used to measure the effects, if any, of unnatural amino acid substitutions on ligand binding. Binding data involving the wild-type versus the altered receptor are compared to define the molecular forces involved in receptor/ligand binding.

The interaction of acetylcholine with the nicotinic acetylcholine receptor has recently been studied in order to develop the receptophore model for the interactions of the nicotinic agonists described in Zhong et al. (1998) Proc. Natl. Acad. Sci. 95:12088-12093. A clear agonist receptophore model of the nicotinic receptor family will emerge after multiple agonist contact points are identified through systematic mapping of the target binding sites using the in vivo nonsense suppression method for unnatural amino acid incorporation. A number of aromatic amino acids have been identified as contributing to the agonist binding site, suggesting that cation-π interactions may be involved in binding the quaternary ammonium group of the agonist, acetylcholine. A compelling correlation has been shown between (i) ab initio quantum mechanical predictions of cation-π binding abilities and (ii) EC₅₀ values for acetylcholine at the receptor for a series of tryptophan derivatives that were incorporated into the receptor by using in vivo nonsense suppression method for unnatural amino acid incorporation. Such a correlation is seen at one, and only one, of the aromatic residues: tryptophan-149 of the α subunit. This finding indicates that, on binding, the cationic, quaternary ammonium group of acetylcholine makes van der Waals contact with the indole side chain of the α tryptophan-149, providing the most precise structural information to date on this receptor. Upon similar systematic probing of other potential steric and electronic interactions at the acetylcholine binding site, a receptophore model will be built for binding and physiological activity of agonists at the nicotinic receptor. This general methodology can be used to build receptophore models for other agonist, antagonist or allosteric interactions with a wide range of receptors and ion channels. Id.

An electrophysiological method such as, for example, the voltage clamp is used to assess the ligand-binding capabilities of altered ion channel receptors. The voltage clamp assay measures ligand binding to a receptor by detecting changes in the membrane potential that are induced by ion transport across the cell membrane. Such electrophysiological methods are well known in the art and have been used extensively for the study of ion channels in the Xenopus oocyte expression system.

Other ligand-binding assays can be developed to measure ligand-receptor binding events that do not involve changes in membrane potential. While a skilled artisan is capable of selecting a biochemical assay for use with a particular expression system, unnatural amino acid, receptor, ligand, and modulator involved in a particular study, provided herein are non-limiting exemplary ligand-binding assays. The invention is not limited by the particular binding assay employed.

In one embodiment, a labeled ligand is used to physically detect the presence of the bound or unbound ligand. Various types of labels, including but not limited to radioactive, fluorescent, and enzymatic labels, have been used in binding studies and are well known in the art. Labeled ligands can be commercially obtained or prepared using techniques known in the art. A binding assay using a radioactively labeled ligand may include the following steps: (1) incubating purified receptors or cells expressing receptors with the labeled ligand, (2) allowing an appropriate time for ligand-binding, (3) counting the number of bound ligands using a scintillation counter, and (4) comparing the differences in radioactive counts for altered and unaltered receptors.

Receptor/ligand binding data are compiled to create a model of a receptor/ligand binding event. The contribution of specific amino acid side chains to ligand binding is inferred from the comparative properties of a natural amino acid with the substituted unnatural amino acid. Therefore, the production of meaningful data will depend in part on the selection of appropriate substitutions. While one skilled in the art is capable of selecting an unnatural amino acid substitution to investigate a putative receptor/ligand interaction, the following provides some examples of how relevant information is extrapolated from these experiments. The invention, however, is not bound by the theories provided below, which are offered to improve the understanding of the invention and its use.

(1) A cation-π interaction is important if fluoro-, cyano-, and bromo- amino acid derivatives, substituted for natural aromatic amino acids, abrogate ligand binding. When incorporated into an aromatic amino acid, these substituents withdraw electron density from the aromatic ring, weakening the putative electrostatic interaction between a positively charged group on the ligand and the aromatic moiety. Fluoro-derivatives are often preferred because fluorine is a strong electron-withdrawing group, and often adds negligible stenc perturbations.

(2) Hydrophobic interactions at a given position are important if ligand binding is affected by substitutions that increase hydrophobicity without significantly altering the sterics of the side chain, thereby allowing the importance of hydrophobic interactions to be investigated in the absence of artificial steric constraints. One example of such a manipulation is conversion of a polar oxygen to a nonpolar CH₂ group, as in O-Methyl-threonine to isoleucine. Other methods to increase hydrophobicity, such as increasing side chain length, as in the substitution of allo-isoleucine for valine, or β-branch addition, as in the substitution of norvaline for isoleucine, or γ-branch addition, as in the substitution of t-butylalanine for isoleucine, may produce results that support the importance of hydrophobic interactions.

(3) A local α-helix or β-sheet structure is important if an α-hydroxy acid substitution influences ligand binding. Incorporation of an a-hydroxy acid into the peptide backbone will produce an ester linkage instead of an amide bond. Since the amide hydrogen bond is important for stabilization of local α-helices and β-sheets, the α hydroxy acid substitution disrupts these structures.

(4) By incorporating the phosphorylated or glycosylated analogue of a given amino acid into the receptor, the skilled artisan can compare ligand binding in the presence or absence of the putative modification.

(5) Using photoreactive unnatural amino acids, the importance of specific side chains or protein modifications can be studied. For example, addition of the photoremovable nitrobenzyl group to the side chain of an amino acid can prevent interactions with the ligand or block side chain modifications such as phosphorylation and methylation. UV irradiation removes the nitrobenzyl group thereby restoring the amino acid to its native form. Therefore, ligand-binding measurements taken before and after UV irradiation can uncover side chain contributions to ligand binding. Similarly, the importance of local protein structures such as loops can be investigated by incorporating the unnatural amino acid (2-nitrophenyl) glycine (Npg). Irradiation of the Npg-modified amino acid triggers proteolysis of the protein receptor backbone. If UV irradiation disrupts ligand binding to the Npg-modified receptor, a structure near the incorporated unnatural amino acid is likely important.

(6) Fluorescent reporter groups such as the nitrobenzoxadiazole (NBD) fluorophore or spin labels such as nitroxyl can be incorporated into the receptor using unnatural amino acids containing these labels. For example, after incorporation of an NBD-amino acid into the receptor, fluorescence resonance energy transfer between a fluorescence-labeled ligand and the NBD-amino acid can provide information such as the distance between the amino acid residue and the ligand binding site.

Identifying and Refining Compounds Specific for Receptor Subtypes and/or Conformations using the Receptophore Model

The invention can be used to develop compounds that are specific for a receptor subtype and/or conformation. First, developing receptophore models for the interactions between receptor subtypes and a ligand that exhibits subtype specificity can identify amino acids that contribute to a ligand's subtype-selective binding. Second, developing receptophore models with receptors that assume different conformations and compounds that stabilize or are selective for a particular conformation can identify conformation-specific contacts. The data from these experiments can be used to engineer a more optimal compound by modifying the compound to take better advantage of subtype-specific or conformation-specific interactions. Although a skilled artisan is capable of predicting chemical alterations that will increase a compound's affinity for a particular subtype and/or conformation, the following provide some examples of this process here. Again, the invention is not bound by the theories provided below, which are offered to improve the understanding of the invention and its use.

(1) If the receptor/ligand model predicts stacking of an aromatic amino acid and an aromatic group of the ligand, a more parallel geometry between the aromatic groups may strengthen this interaction.

(2) If the receptor/ligand model suggests the importance of a specific hydrogen bond, a stronger hydrogen-bonding group can be substituted to increase the ligand's affinity for the receptor.

(3) If the ligand contains groups that sterically hinder its binding, these groups can be removed in favor of smaller groups.

(4) If hydrophobic forces contribute to the interaction at a particular position, less polar or larger hydrocarbon groups can be substituted within the steric limitations of the binding site.

If an aromatic group in the binding site is left unengaged by the inhibitor, a positively charged group in the appropriate geometry for a cation-π interaction may increase the compound's affinity for the binding site.

Having now generally described the invention, the same will be more readily understood through reference to the following examples which are provided by way of illustration, and are not intended to be limiting of the present invention, unless specified.

### EXAMPLES

Materials

Synthetic DNA oligonucleotides were synthesized on an ABI 394 DNA Synthesizer. Restriction enzymes and T4 RNA Ligase were purchased from New England Biolabs (Beverly, Mass.). The mMessage mMachine and MegaShortScript in vitro trancription kits were purchased from Ambion (Austin, Tex.). Maxiprep kits used for plasmid isolation were purchased from Qiagen (Valencia, Calif.). Two membrane localized GFP mammalian expression vectors were used, pCS2gapEGFP (Jack Home, Caltech) and pEGFP-F (Clontech, Palo Alto, Calif.). pEGFP-N1, a soluble GFP construct, was also purchased from Clontech (Palo Alto, Calif.). Ham's F12 tissue culture media was purchased from Irvine Scientific (Santa Ana, Calif.), and CO₂ independent and Neurobasal Media were purchased from Gibco BRL (Grand Island, N.Y.). The microporator was built on site.

Mutagenesis, mRNA Synthesis and tRNA Synthesis

The HSAS gene was constructed as follows: two complementary synthetic oligos encoding for the T7 promoter, the HSAS gene, and the Fok1 restriction site were annealed and ligated into the EcoR1 and BamH1 restriction sites of pUC19 (Nowak et al., 1998). Linearization with Fok1 yields 74 mer tRNA upon in vitro transcription with the MegaShortScript kit (i.e., lacking the 3′ terminal CA nucleotides).

THG73 tRNA (Nowak et al., 1998), THG73-W and THG73-F₂W (Zhong et al., 1998) have been previously described in the art. Briefly, linearization of pUC19 containing the THG73 gene with Fok1 yields 74 mer tRNA upon in vitro transcription with the MegaShortScript kit. The THG73 74 mer was then ligated to dCA-W or dCA-F₂W with T4 RNA ligase to generate THG73-aa.

EGFP mutants (pCS2gapEGFP and pEGFP-F Ser29TAG) and nAChR β subunit mutants (Leu9′Ser and Leu9′TAG) were made following the Quickchange mutagenesis protocol (Stratagene).

The mRNA that encodes for the muscle type nAChR subunits (α, β, β and γ) was obtained by linearization of the expression vector (pAMV) with Not1, followed by in vitro transcription using the mMessage mMachine kit.

Unnatural Amino Acids

While most unnatural amino acids were purchased from commercial sources, other unnatural amino acids can be synthesized by known techniques. Tryptophan analogues were prepared using the method of Gilchrist et al. (1979) J. Chem. Soc. Chem. Commun. 1089-90. Tetrafluoroindole was prepared by the method of Rajh et al. (1979) Int. J. Pept. Protein Res. 14:68-79. 5, 7-Difluoroindole and 5,6,7-trifluoroindole were prepared by the reaction of CuI/dimethylformamide with the analogous 6-trimethylsilylacetylenylaniline.

Typically, the amino group was protected as the o-nitroveratryloxycarbonyl (NVOC) group, which was subsequently removed photochemically according to methods known in the art. However, for amino acids that have a photoreactive sidechain, an alternative, such as the 4-pentenoyl (4PO) group, a protecting group first described by Fraser-Reid, was used. Madsen et al. (1995) J. Org. Chem. 60, 7920-7926; Lodder et al. (1997) J. Org. Chem. 62, 778-779. Presented herein is a representative procedure based on the unnatural amino acid (2-nitrophenyl)glycine (Npg), as described in England, et al. Proc. Natl. Acad. Sci. USA (in press).

N-4PO-D,L-(2-nitrophenyl)glycine. The unnatural amino acid D,L-(2-nitrophenylglycine)hydrochloride was prepared according to Davis et al. (1973) J. Med. Chem. 16, 1043-1045; Muralidharan et al. (1995) J. Photochem. Photobiol. B: Biol. 27, 123-137. The amine was protected as the 4-pentenoyl (4PO) derivative as follows. To a room temperature solution of (2-nitrophenyl)glycine hydrochloride (82 mg, 0.35 mmol) in H₂O:dioxane (0.75 ml:0.5 ml) was added Na₂CO₃ (111 mg, 1.05 mmol), followed by a solution of 4-pentenoic anhydride (70.8 mg, 0.39 mmol) in dioxane (0.25 ml). After 3 hours the mixture was poured into saturated NaHSO₄ and extracted with CH₂CI₂. The organic phase was dried over anhydrous Na₂SO₄ and concentrated in vacuo. The residual oil was purified by flash silica gel column chromatography to yield the title compound (73.2 mg, 75.2%) as a white solid. ¹H NMR (300 MHz, CD₃OD) δ 8.06 (dd, J=1.2, 8.1 Hz, 1H), 7.70 (ddd, J=1.2, 7.5, 7.5 Hz, 1H), 7.62-7.53 (m, 2H), 6.21 (s, 1H), 5.80 (m, 1H), 5.04-4.97 (m, 2H), 2.42-2.28 (m, 4H). HRMS calcd. for C₁₃H₁₄N₂O₅ 279.0981, found 279.0992.

N-4PO-D,L-(2-nitrophenyl)glycinate cyanomethyl ester. The acid was activated as the cyanomethyl ester using standard methods known in the art. Robertson et al. (1989) Nucleic Acids Res. 17, 9649-9660; Ellman et al. (1991) Meth. Enzym. 202, 301-336. To a room temperature solution of the acid (63.2 mg, 0.23 mmol) in anhydrous DMF (1 ml) was added NEt₃ (95 μl, 0.68 mmol) followed by C1CH₂CN (1 ml). After 16 hours the mixture was diluted with Et₂O and extracted against H₂O. The organic phase was washed with saturated NaC1, dried over anhydrous Na₂SO₄, and concentrated in vacuo. The residual oil was purified by flash silica gel column chromatography to yield the title compound (62.6 mg, 85.8%) as a yellow solid. ¹H NMR (300 MHz, CDC1₃) δ 8.18 (dd, J=1.2, 8.1 Hz, 1H), 7.74-7.65 (m, 2H), 7.58 (ddd, J=1.8, 7.2, 8.4 Hz, 1H), 6.84 (d, J=7.8 Hz, 1H), 6.17 (d, J=6.2 Hz, 1H), 5.76 (m, 1H), 5.00 (dd, J=1.5, 15.6 Hz, 1H), 4.96 (dd, J=1.5, 9.9 Hz, 1H), 4.79 (d, J=15.6 Hz, 1H), 4.72 (d, J=15.6 Hz, 1H), 2.45-2.25 (m, 4H). HRMS calcd. for C₁₆H₁₇N₃O₅ 317.1012, found 317.1004.

N-4PO-(2-nitrophenyl)glycine-dCA. The dinucleotide dCA was prepared as reported by Schultz (Id.) with the modifications described by Kearney et al. (1996) Mol. Pharmacol. 50, 1401-1412. The cyanomethyl ester was then coupled to dCA as follows. To a room temperature solution of dCA (tetrabutylammonium salt, 20 mg, 16.6 μmol) in anhydrous DMF (400 μl) under argon was added N-4PO-D,L-(2-nitrophenyl)glycinate cyanomethyl ester (16.3 mg, 51.4 μmol). The solution was stirred for 1 hour and then quenched with 25 mM NH₄OAc, pH 4.5 (20 μl). The crude product was purified by reverse-phase semi-preparative HPLC (Whatman Partisil 10 ODS-3 column, 9.4 mm×50 cm), using a gradient from 25 mM NH₄OAc, pH 4.5 to CH₃CN. The appropriate fractions were combined and lyophilized. The resulting solid was redissolved in 10 mM HOAc/CH₃CN and lyophilized to afford 4PO-Npg-dCA (3.9 mg, 8.8%) as a pale yellow solid. ESI-MS⁻ M 896 (31), [M-H]⁻ 895 (100), calcd for C₃₂H₃₆N₁₀O₁₇P₂ 896. The material was quantified by UV absorption (ε≈37,000 M⁻¹ cm⁻¹).

Tissue Culture

CHO cells and HEK cells were grown at 37° C. and 5% CO₂ in Ham's F12 media, enriched with glutamine, fetal bovine serum (FBS, 10%), penicillin and streptomycin. The cells were passaged 1 to 2 days prior to electroporation, such that they were typically 50% or less confluent, grown in 35 mm tissue culture dishes.

Rat E18 hippocampal neurons were prepared using procedures known in the art. Li et al., J. Neurosci. 1998, 18: 1023 1-10240. Briefly, hippocampi were digested with 0.25% trypsin and then triturated. Cells plated in polylysine coated 35 mm plastic dishes were maintained in Neurobasal medium supplemented with B27, 500 μM Glutamax, and 5% horse serum (Invitrogen). Transfections were done after 5 days in culture.

Electroporation

The DNA, mRNA or tRNA to be electroporated into CHO cells, HEK cells, or neurons was precipitated alone or as co-precipitates in ethanol and ammonium acetate, and left at −20° C. for at least one hour. For THG73-aa, the amino acids were protected with NVOC at the N-terminus, and were photo-deprotected immediately prior to electroporation. This consisted of irradiating a 15 μl solution of THG73-aa (1 μg/μl) in 1 mM NaOAc (pH 4.5) for 6 minutes, using a 1000 W Hg/Xe arc lamp (Oriel) operating at 400 W, equipped with WG355 and UG11 filters (Schott). Reporter EGFP DNA and nAChR subunit mRNA were then combined with this and precipitated with ammonium acetate and ethanol. This was then centrifuged at 4° C. for 15 minutes, vacuum dried for 5 minutes, and resuspended in CO₂ independent medium to the desired final concentration. Immediately prior to electroporation, the cell tissue culture media was swapped to CO₂ independent media (with no glutamine, FBS or antibiotics). Approximately 5 μl of the electroporation solution was applied to the cells followed by application of electrical pulses. For CHO cells, this was typically four 120 V pulses of 50 ms duration, and for neurons, four 150 V pulses of 25 ms duration. The CO₂ independent media was immediately replaced with fresh Ham's F12 for CHO cells, or the original neurobasal media for neurons, and the cells were placed back into the 37° C. incubator. Imaging of EGFP was done as soon as 2 hours after transfection, and electrophysiogical recordings were done 24 hours after transfection.

CHO cells and neurons were typically transfected with EGFP DNA (2 μg/μl) with or without HSAS tRNA (4 μg/μl). CHO cells were transfected with nAChR mRNA for each of the subunits α (1 μg/μl), β, δ, and γ (0.5 μg/μl each), with or without HSAS tRNA (2 μg/μl or THG73-aa (4 μg/μl) and wt pEGFP-N1 DNA (0.5 μg/μl).

Microscopy

CHO cells and neurons were visualized with an inverted microscope (Olympus IMT2), a 250 W Hg/Xe lamp operating at 150 W, a GFP filter set (Chroma, model 41017) with an excitation band pass of 450 to 490 nm and an emission band pass of 500 to 550 nm, 10×/0.25NA or 40×/1.3NA lens and a Photometrix Quantix CCD camera running Axon imaging Workbench 4.0.

Electrophysiology

Whole cell recordings were performed on EGFP expressing cells. The cells were visualized using an inverted microscope as described above. Patch electrodes (borosilicate, 4-6 MΩ) were filled with a pipette solution containing: 88 mM KH₂PO₄, 4.5 mM MgC1₂, 0.9 mM EGTA, 9 mM HEPES, 0.4 mM CaC1₂ 14 mM creatine phosphate, 4 mM Mg-ATP, 0.3 mM GTP (Tris salt), adjusted to pH 7.4 with KOH. The recording solution contained: 150 mM NaCI, 4 mM KC1, 2 mM CaC1₂, 2 mM MgC1₂, 10 mM glucose, 10 mM HEPES, and 1 μM atropine, adjusted to pH 7.4 with NaOH. Standard whole cell recordings were done using an Axopatch 1-D amplifier, low-pass filtered at 2-5 kKz and digitized online at 20 kHz (pClamp 8, Foster City, Calif.). The membrane potential was held at −60 mV.

Acetylcholine (ACh) was delivered using a two-barrel glass theta tube (outer diameter ˜200 μm, pulled from 1.5 mm diameter theta borosilicate tubing) connected to a piezo electric translator (Burleigh LSS-3100, Fisher, N.Y.). Each barrel of the theta tube was fed from a twelve-way manifold. This allowed up to twelve different solutions to be fed in either the control or agonist barrel. Agonists were applied for 25 ms, which was triggered by pClamp 8 software. The voltage input to the high voltage amplifier (Burleigh PZ-150M, Fishers, N.Y.) used to drive the piezo translator was filtered at 150 Hz by an 8-pole Bessel filter (Frequency Devices, Haverhill, Mass.), to reduce oscillations from rapid pipette movement. Solution exchange rates measured from open tip junction potential changes upon application with 10% recording solution were typically ˜300 μs (10-90% peak time).

Whole-cell current responses to various ligand concentrations at indicated holding potentials (typically −60 mV) were fitted to the Hill equation, I/Iₘₐₓ=1/{1+(EC₅₀/[A])ⁿ}, where I is agonist-induced current at [A], Iₘₐₓ is the maximum current, EC₅₀ is the concentration inducing half-maximum response, and n is the Hill coefficient.

Expression of EGFP by Nonsense Suppression

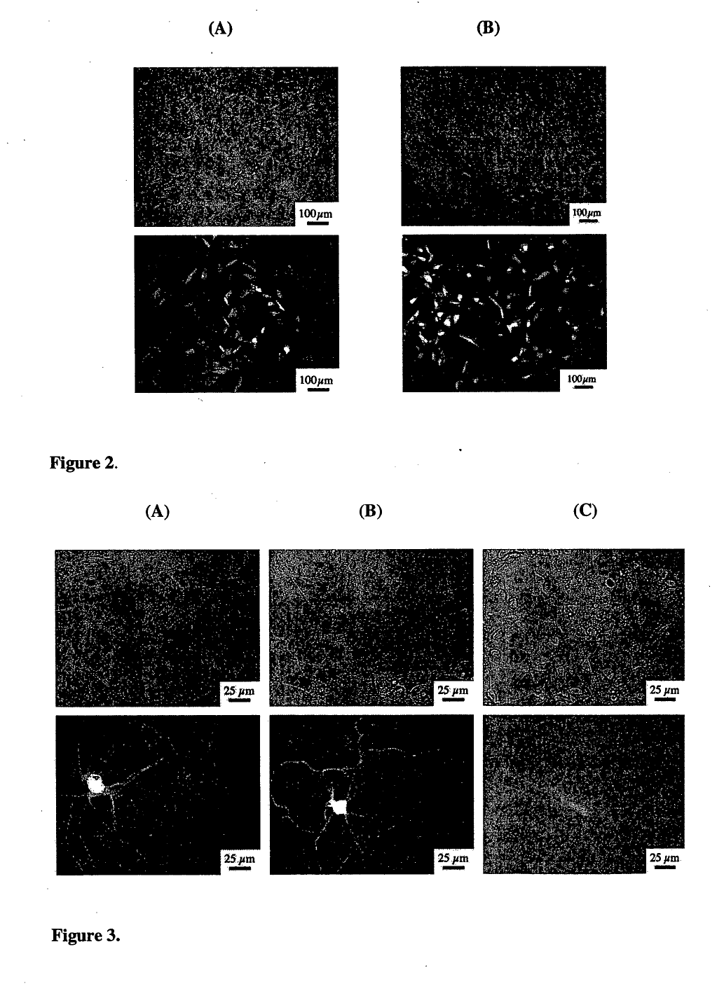

The EGFP DNA gene was mutated to introduce a TAG stop codon at Ser29. This DNA was combined with in vitro transcribed HSAS to a volume of 5 μl. Transfection was achieved by applying this solution to adherent CHO-K1 cells and applying four 120 V, 50 ms square wave pulses. As can be seen in FIG. 2, two hours after transfection there is high EGFP expression in cells transfected with either wild type pEGFP-F, or mutant S29TAG pEGFP-F DNA and HSAS. When only the S29TAG mutant DNA is transfected without the HSAS, no EGFP expression is observed. This demonstrates that both DNA and tRNA can be co-electroporated into cells with high efficiency. Furthermore, this shows that in vitro transcribed HSAS is aminoacylated by the endogeneous CHO-K1 synthetase and is a fully functional suppressor tRNA in mammalian cells.

Nonsense Suppression in Hippocampal Neurons

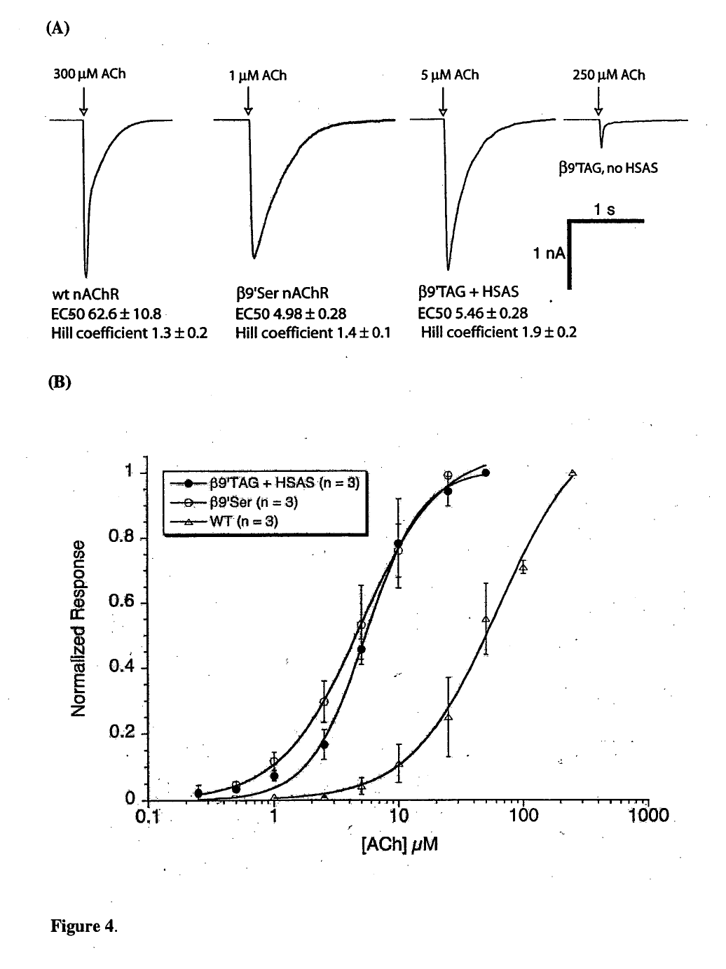

In order to demonstrate the generality of the method of the present invention, the EGFP suppression assay was tested in neurons. As for CHO transfection, HSAS tRNA and Ser29TAG EGFP DNA were co-electroporated into E18 rat hippocampal neurons (5 days in culture). As can be seen in FIG. 3, 24 hours after transfection EGFP suppression by HSAS leads to comparable expression levels as electroporation of wild type EGFP DNA. This demonstrates that electroporation also efficiently delivers tRNA and DNA to neurons, and that the neurons also readily use in vitro transcribed tRNA for nonsense suppression. As shown in FIG. 3C, only low levels of fluorescence were detected when no tRNA is added, indicating minimal read-through of the Ser29TAG construct.

Expression of the Muscle Type nAChR by Nonsense Suppression

Previous experiments with the nAChR involved the mutation of a Leu residue in the M2 pore lining region, termed Leu9′ (Charnet et al., Neuron 1990, 4: 87-95), that is conserved in all known nAChR subunits. Earlier studies showed that nAChR with a Leu9′Ser mutation exhibits increased sensitivity to ACh, and consequently a characteristic decrease in the EC₅₀. For example, the L9′S mutation of the β subunit leads to a decrease in the EC₅₀ by an order of magnitude from that of the wild type channel. Kearney et al., Neuron 1996, 17: 1221-1229; Labarca et al., Nature 1995, 376: 514-5 16. Therefore, suppression of the βL9′TAG by HSAS should lead to expression of ion channels that display a substantial shift in the dose-response curve, the characteristic βL9′S phenotype.

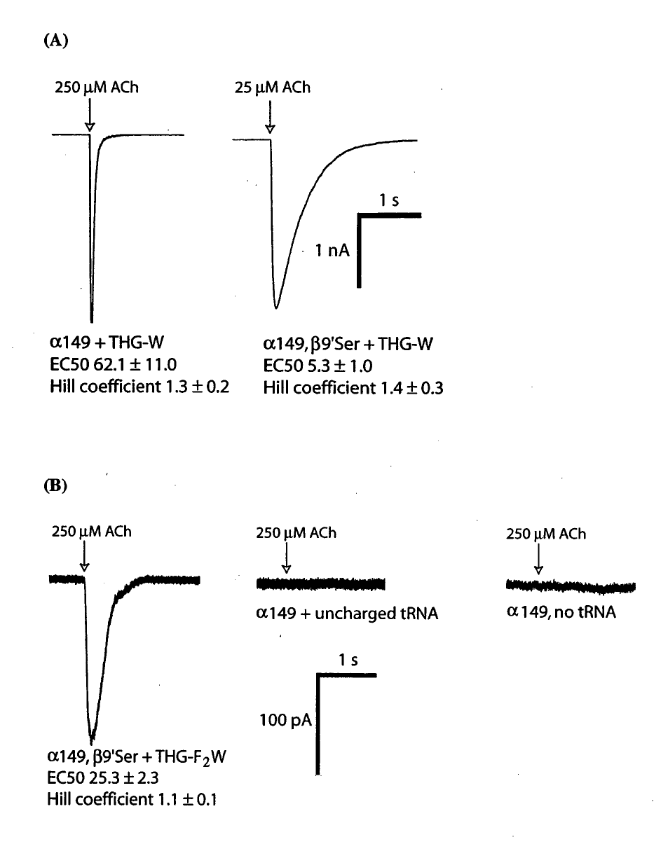

Transfection of CHO-K1 or HEK cells was achieved by electroporation of a 5 μl solution containing HSAS, mutant β subunit mRNA (L9′TAG), and mRNA for the remaining wild type subunits (α, γ, and δ). Also included was a reporter EGFP plasmid. Expression of the nAChR was determined from whole cell recordings of EGFP-expressing CHO-K1 cells upon ACh application. As shown in FIG. 4, 24 hours after transfection the cells exhibit a strong ACh response that is not observed in nontransfected cells. Both the receptors generated from HSAS-suppressed β9′TAG mutant mRNA and from the β9′Ser mutant showed substantial shifts in their dose-response curves relative to wild type. This demonstrates that HSAS did indeed deliver serine during translation of the βL9′TAG subunit, as is apparent from the phenotype. Importantly, the fact that the magnitude of the ACh response is the same for the HSAS suppressed channels as for both the wild type and βL9′S nAChR demonstrates that ion channel expression is not tRNA limited, validating that electroporation leads to highly efficient delivery of tRNA to the cells.

Unnatural Amino Acid Incorporation in CHO Cells

Extensive studies of the agonist binding site of the nAChR established a critical role for Trp α149 in agonist binding (Zhong et al., 1998). The most telling evidence for this arises from substitution of fluorinated Trp derivatives, which produce a systematic shift in the EC₅₀ for ACh activation. In these initial studies, the THG73 amber suppressor proved to be very effective for studies in Xenopus oocytes (Saks et al., J. Biol. Chem. 1996, 271).

Using the electroporation protocol of the present invention, mutant α subunit mRNA (TAG 149), mRNA for the remaining subunits (β with the L9′S mutation, γ, and δ), and a reporter EGFP plasmid were delivered to CHO cells. Also included was the tRNA THG73 that had been chemically aminoacylated with either Trp (wild type, THG-W) or 5,7-difluorotryptophan (THG-F₂W).

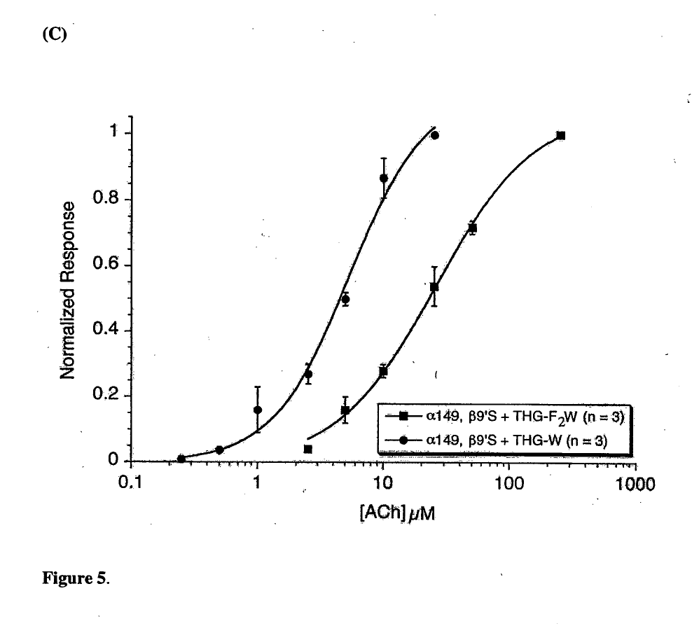

When evaluated 24 hours after transfection, currents as large as 2 nA (range from 100 pA to 2 nA) in response to ACh are seen. As shown in FIG. 5, when THG73 is used to deliver Trp, a wild type channel is produced. Most importantly, ThG73 aminoacylated with F₂W leads to a characteristic shift in the dose-response curve to higher EC₅₀. Control experiments using THG73 that has not been aminoacylated gave no current.

Development of Receptophore Model

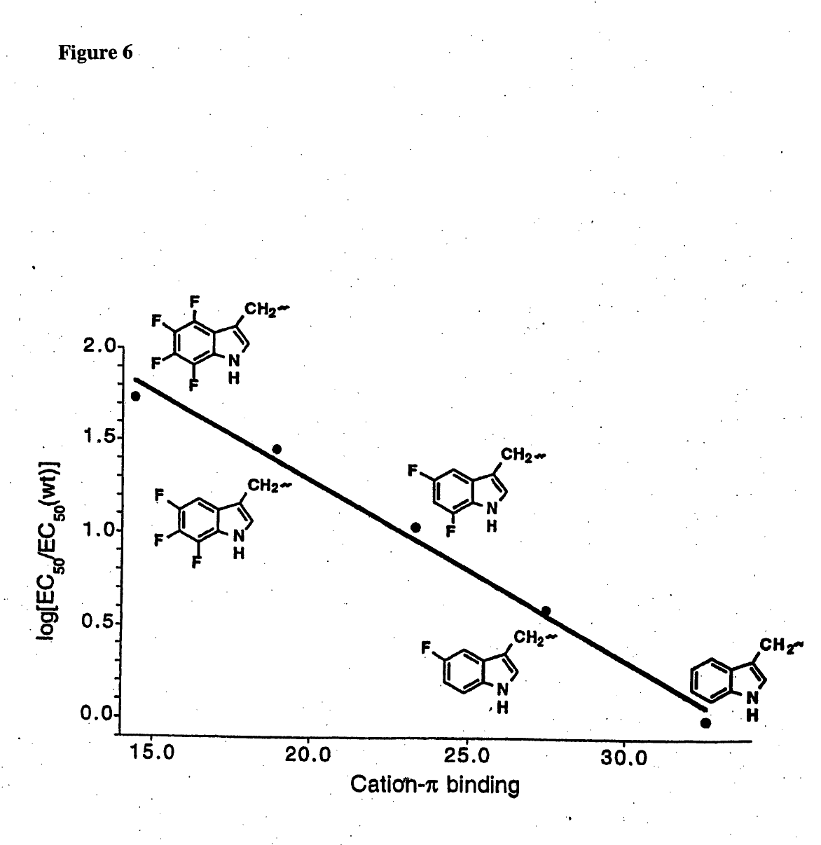

Dose-response curves were fitted to the Hill equation for the unaltered receptor (WT) and for unnatural amino acid substitutions at α-Trp 149. Substitutions include 5-F-Trp, 5,7-F₂-Trp, 5,6,7-F₃-Trp and 4,5,6,7-F₄-Trp. The log[EC₅₀/EC_{40(wt)}] for each the unaltered receptor was plotted vs. cation-π binding ability of each fluorinated ftp derivative. Cation-π binding ability for both trp and the fluorinated derivatives was predicted using ab initio quantum mechanical calculations. Mecozzi et al. (1996) J. Amer. Chem. Soc. 118: 2307-2308; Mecozzi et al. (1996) Proc. Nati. Acad. Sci. USA 93:10566-10571. Data fit the line y=3.2−0.096x, with a correlation coefficient r=0.99. See FIG. 6. These data are consistent with a cation-π bond between α-trp 149 and the quaternary ammonium of acetylcholine in the bound position because each substitution's EC₅₀ value corresponds well with the predicted loss in binding energy due to the substitution. After further systematic mapping of contacts between acetylcholine and the nicotinic acetylcholine receptor, a receptophore model describing the complete steric and electronic features involved in this interaction can be made.

All references cited herein, including patents, patent applications, and publications, are hereby incorporated by reference in their entireties, whether previously specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth.

What is claimed is:

1. A method for the delivery of tRNA along with nucleic acid corresponding to a protein of interest to mammalian cells comprising transfecting cultured mammalian cells with the tRNA and the nucleic acid corresponding to the protein of interest.

2. The method of claim 1 wherein the mammalian cells are transfected by electroporation.

3. The method of claim 1 wherein the tRNA is an in vitro transcript.

4. The method of claim 1 wherein the tRNA is a human amber suppressor tRNA transcript.

5. The method of claim 1 wherein the tRNA is a chemically aminoacylated suppressor tRNA transcript.

6. The method of claim 1 wherein the nucleic acid is DNA or mRNA.

7. The method of claim 1 wherein the protein of interest is a reporter protein.

8. The method of claim 7 wherein the reporter protein is green fluorescent protein (EGFP).

9. The method of claim 1 wherein the protein of interest is a ligand-gated ion channel.

10. The method of claim 9 wherein the ligand-gated ion channel is nicotinic acetylcholine receptor (nAChR).

11. The method of claim 1 wherein the mammalian cells are a cultured mammalian cell line.

12. The method of claim 11 wherein the cultured mammalian cell line is comprised of CHO-K1 cells.

13. The method of claim 11 wherein the cultured mammalian cell line is comprised of HEK cells.

14. The method of claim 1 wherein the mammalian cells are cultured hippocampal neurons.

15. An in vitro tRNA transcript comprising a human amber suppressor tRNA, for delivery to cultured mammalian cells.

16. An in vitro tRNA transcript comprising a chemically aminoacylated suppressor tRNA, for delivery to cultured mammalian cells.

17. A method for the incorporation of unnatural amino acids into mammalian cells, comprising:
a) incorporating a nonsense codon into the nucleic acid corresponding to a protein of interest;
b) producing a tRNA transcript that contains the corresponding anticodon to the nonsense codon;
c) chemically aminoacylating the tRNA transcript with the desired unnatural amino acid; and
d) transfecting cultured mammalian cells with the aminoacyl tRNA and the nucleic acid corresponding to the protein of interest.

18. The method of claim 17 wherein the mammalian cells are transfected by electroporation.

19. The method of claim 17 wherein the tRNA is an in vitro transcript.

20. The method of claim 17 wherein the tRNA is a chemically aminoacylated suppressor tRNA transcript.

21. The method of claim 17 wherein the nucleic acid is DNA or mRNA.

22. The method of claim 17 wherein the protein of interest is a ligand-gated ion channel.

23. The method of claim 22 wherein the ligand-gated ion channel is nicotinic acetyicholine receptor (nAChR).

24. The method of claim 17 wherein the mammalian cells are a cultured mammalian cell line.

25. The method of claim 24 wherein the cultured mammalian cell line is comprised of CHO-K1 cells.

26. The method of claim 24 wherein the cultured mammalian cell line is comprised of HEK cells.

27. The method of claim 17 wherein said unnatural amino acids are represented by the Formula (I): 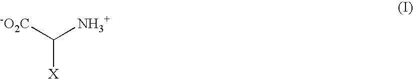
where X is selected from the group consisting of:

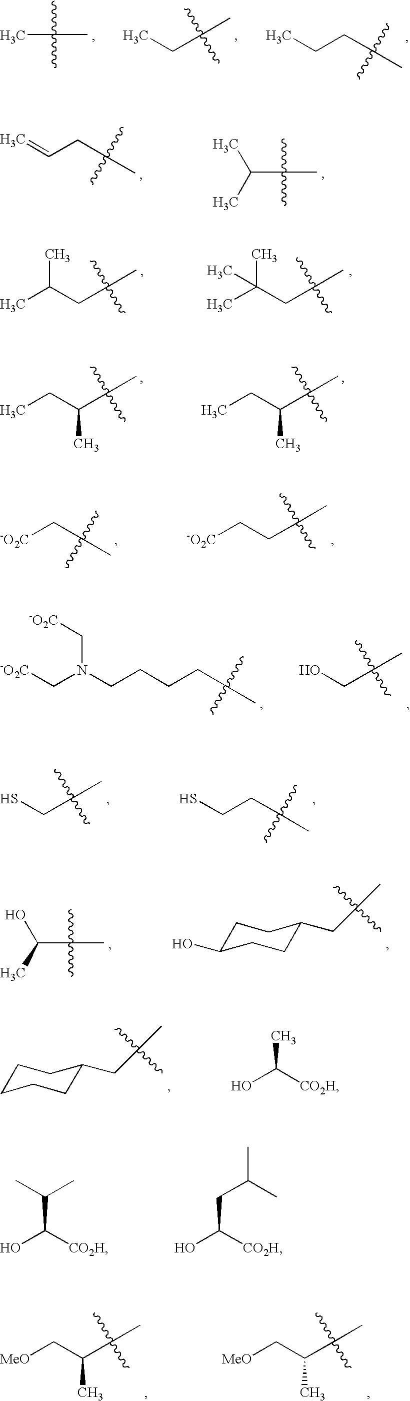

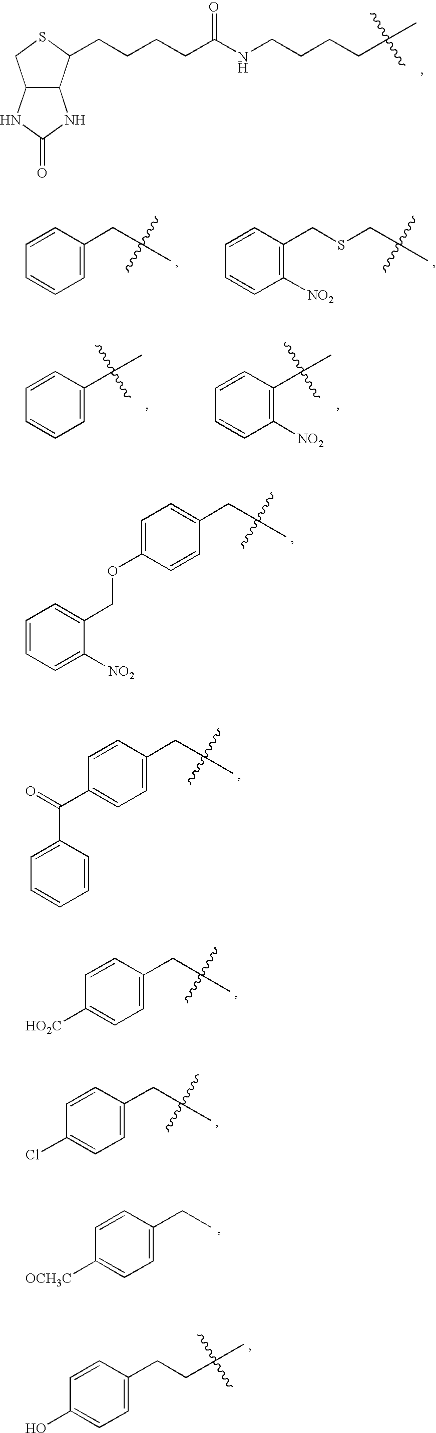

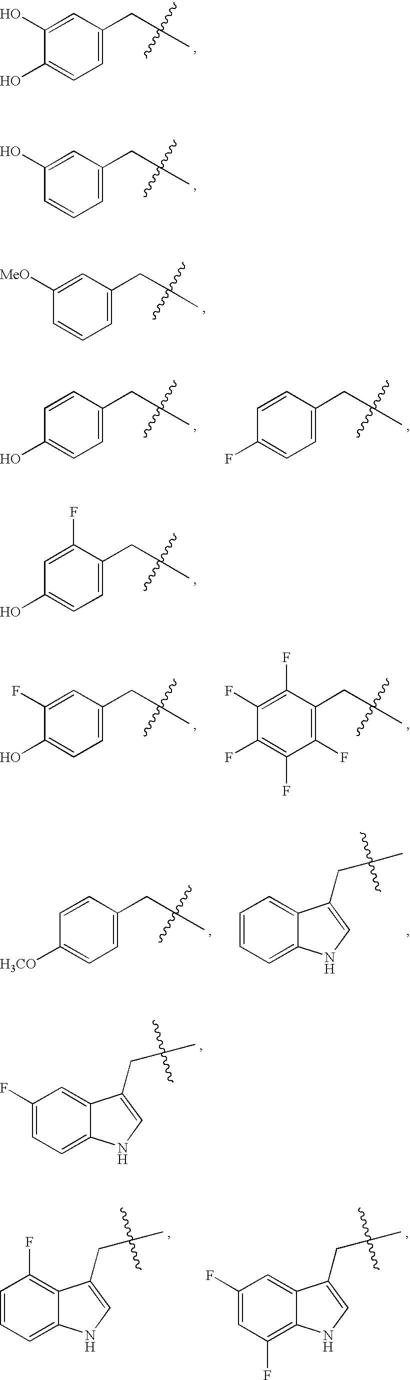

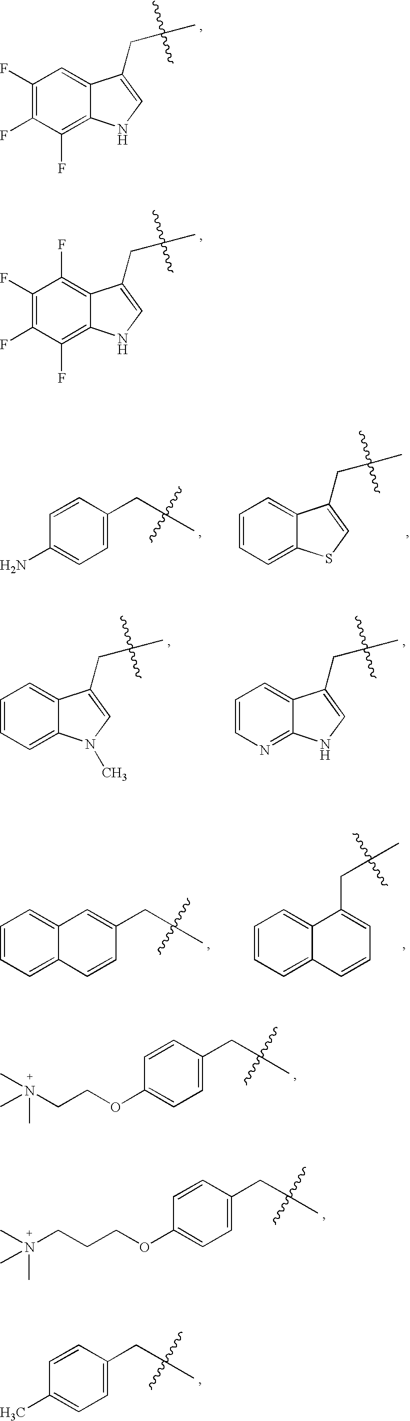

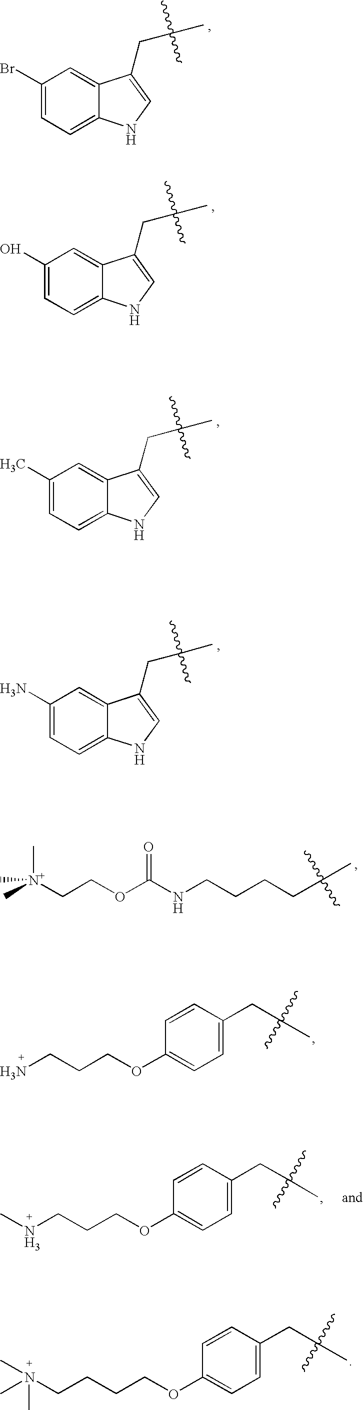

28. The method of claim 17 wherein said unnatural amino acids are represented by the Formula (II): 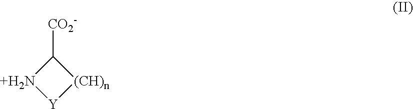
wherein: Y is CH₂, (CH)ₙ, N, O, or S, and n is 1 or 2.

29. The method of claim 17 wherein said unnatural amino acids are selected from the group consisting of:

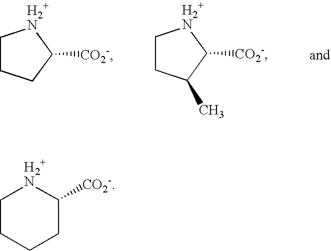

30. An in vitro tRNA transcript comprising a chemically aminoacylated suppressor tRNA that is used for the incorporation of unnatural amino acids into mammalian cells.

31. A method for the incorporation of imaging reagents into mammalian cells, comprising:
a) incorporating a nonsense codon into the nucleic acid corresponding to a protein of interest;
b) producing a tRNA transcript that contains the corresponding anticodon to the nonsense codon;
c) chemically aminoacylating the tRNA transcript with an imaging reagent; and
d) transfecting cultured mammalian cells with the aminoacyl tRNA and the nucleic acid corresponding to the protein of interest.

32. The method of claim 31 wherein the mammalian cells are transfected by electroporation.

33. The method of claim 31 wherein the tRNA is an in vitro transcript.

34. The method of claim 31 wherein the tRNA is a chemically aminoacylated suppressor tRNA transcript.

35. The method of claim 31 wherein the nucleic acid is DNA or mRNA.

36. The method of claim 31 wherein the imaging reagent is a fluorescent amino acid.

37. An in vitro tRNA transcript comprising a chemically aminoacylated suppressor tRNA that is used for the incorporation of imaging reagents into mammalian cells.

38. A method for identifying the specific physiological effect of a compound on the activity of its receptor comprising:
a) a screening method, where said method allows for development of receptophore models;
b) receptophore models based on details of the nature and location of receptor binding of said compounds;
c) receptophore models, wherein said models allow for generation of compounds that can selectively modulate a receptor subtype in a specific receptor conformation to achieve a desired physiological activity; and
d) predicting and designing compounds that could achieve said physiological activity on the target receptor by evaluating how, where, and in what receptor conformation state said compound binds to the receptor.

39. A method for determining the nature of a compound's interaction with a receptor comprising:
a. incorporating unnatural amino acids into binding sites and regulatory sites of the receptor, resulting in an altered receptor;
b. measuring the compound's ability to bind to the altered receptor; and
c. comparing the results of step (b) to the same compound's ability to bind to an unaltered receptor.

40. The method of claim 39 wherein said unnatural amino acids are represented by the Formula (I): 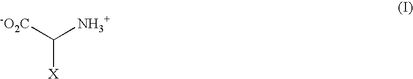
where X is selected from the group consisting of:

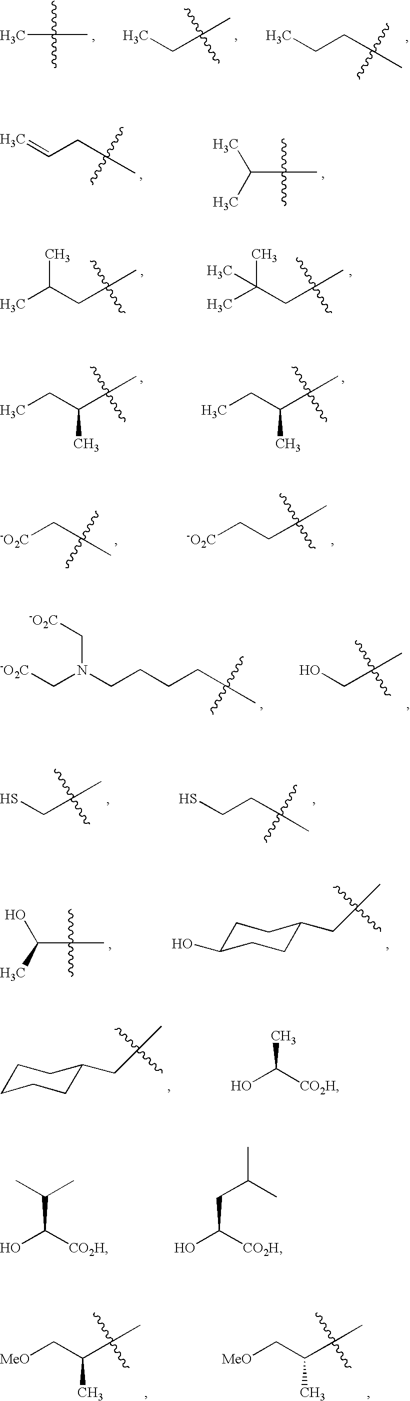

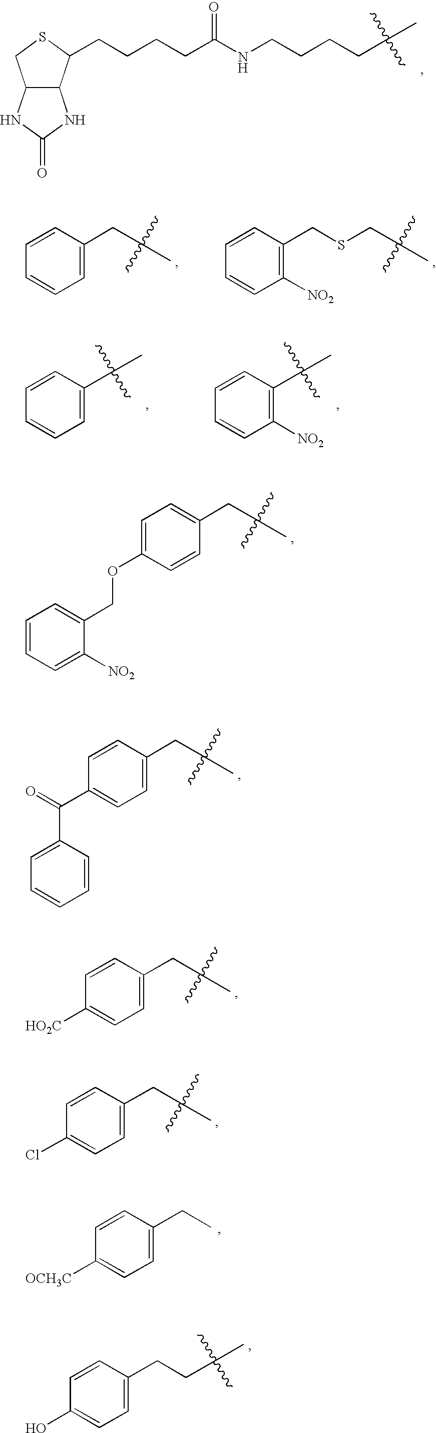

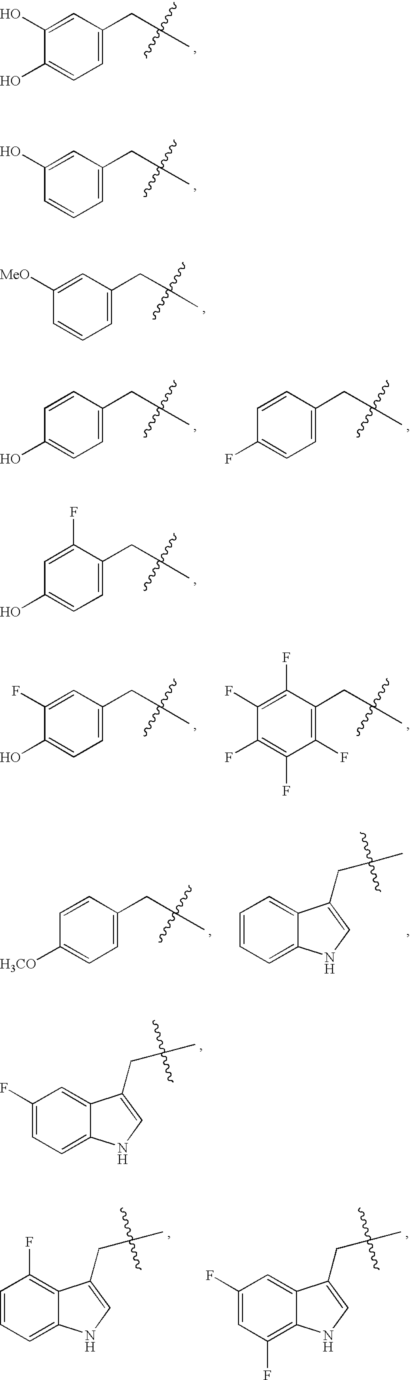

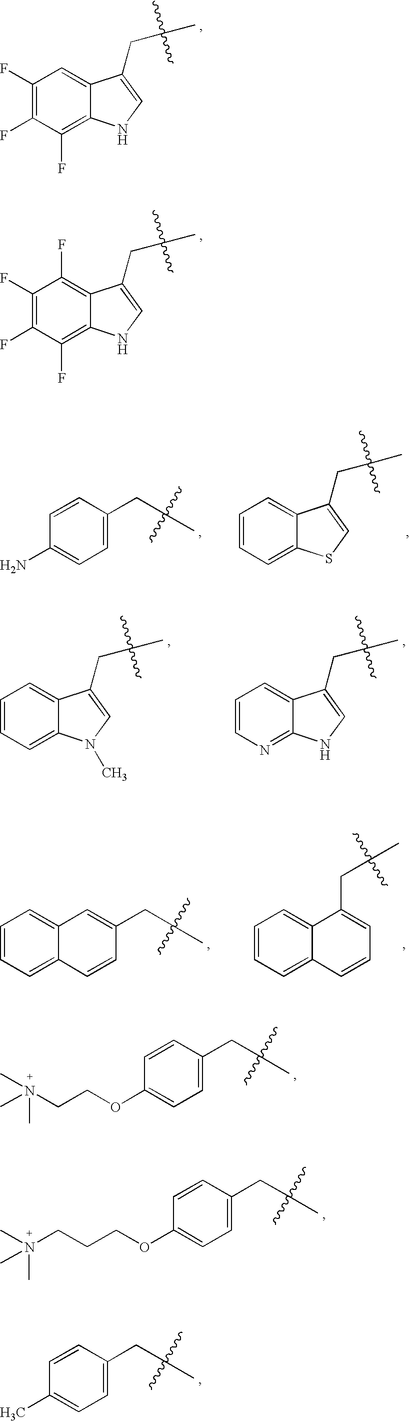

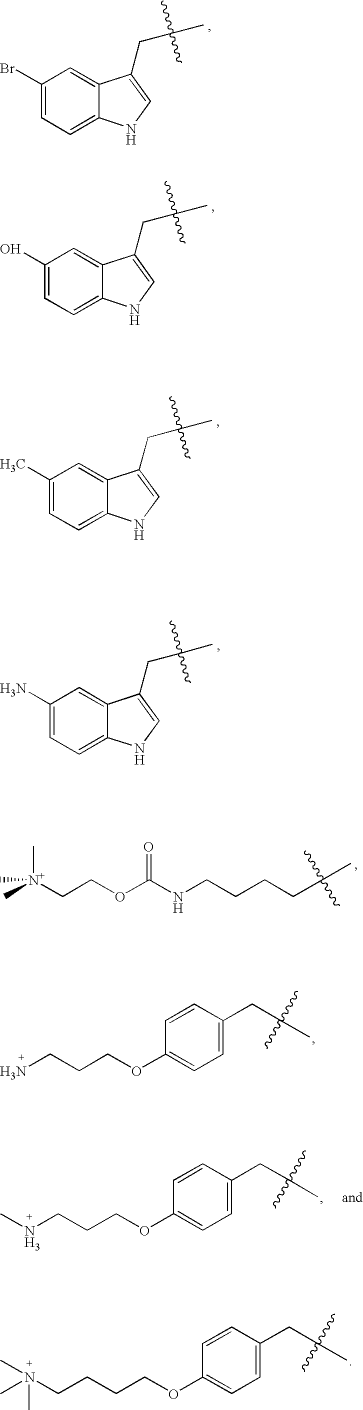

41. The method of claim 39 wherein said unnatural amino acids are represented by the Formula (II): 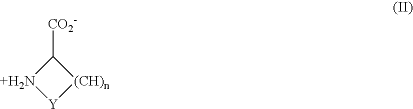
wherein: Y is CH₂, (CH)ₙ, N, O, or S, and n is 1 or 2.

42. The method of claim 39 wherein said unnatural amino acids are selected from the group consisting of:

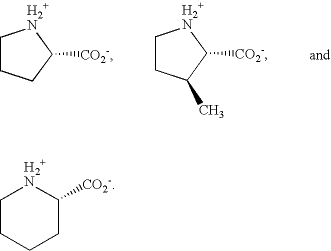

43. The method of claim 39 wherein the receptor is expressed in mammalian cells.

44. A method of identifying key interactions that would lead to discovery of new compounds comprising:
a. determining the nature of the compound's interaction with the receptor;
b. analyzing how and where the compound interacts with the receptor; and
c. based on the analysis in step (b), chemically modifying the compound to achieve desired ligand activity.

45. A screening method comprising a membrane protein receptor which has been modified at the binding or regulatory site to replace native amino acids with unnatural amino acids.

46. The screening method of claim 45 wherein the native amino acids to be replaced are selected from the group consisting of any of the 20 naturally occurring amino acid side chains.

47. The screening method of claim 45 wherein said unnatural amino acids are represented by the Formula (I): 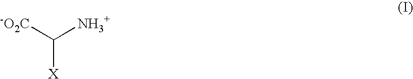
where X is selected from the group consisting of:

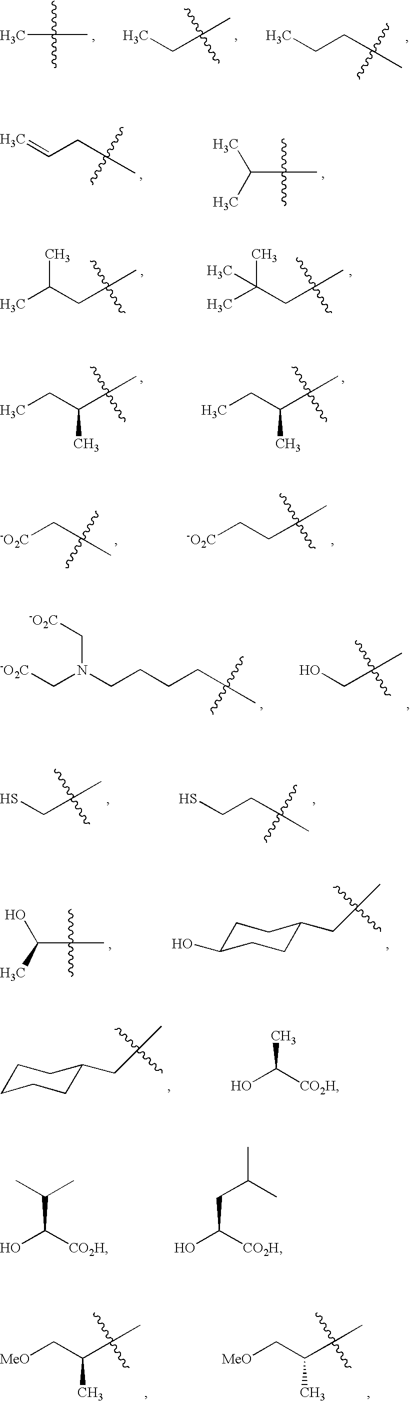

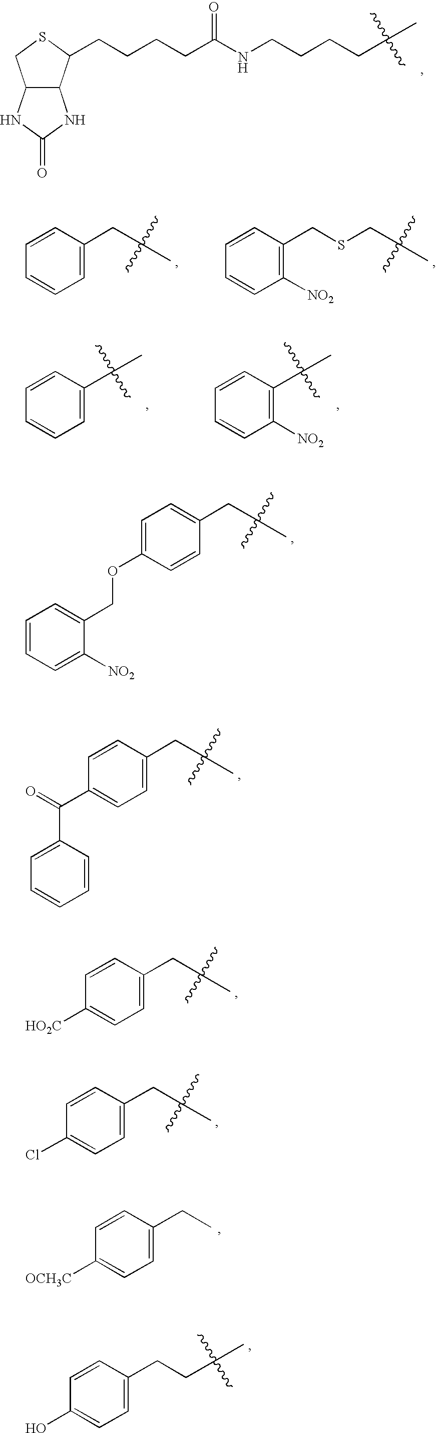

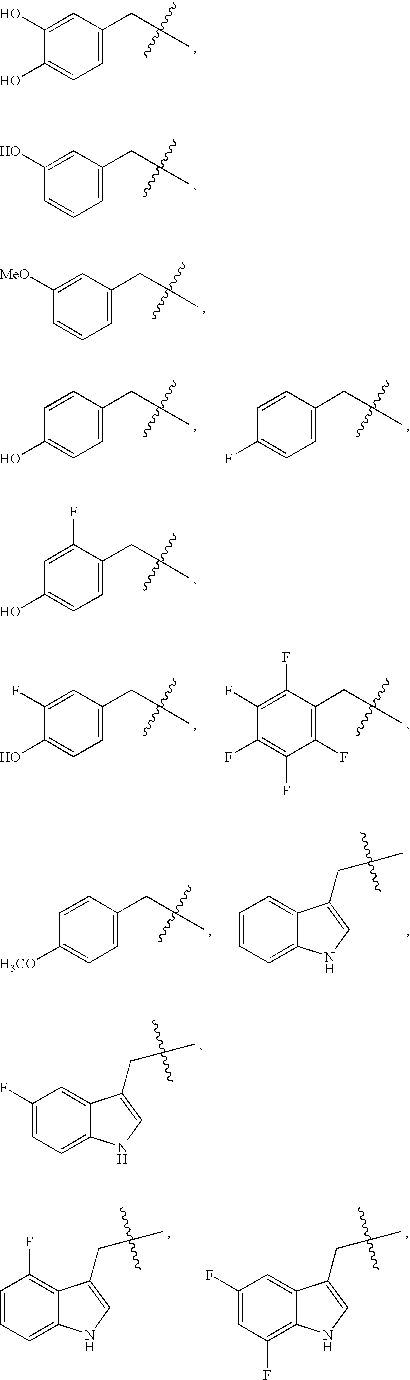

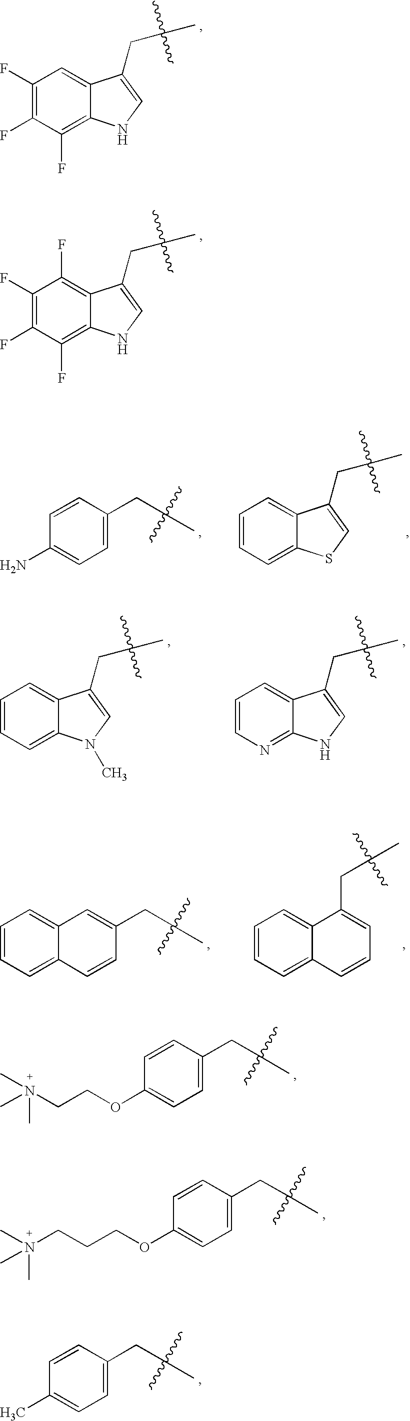

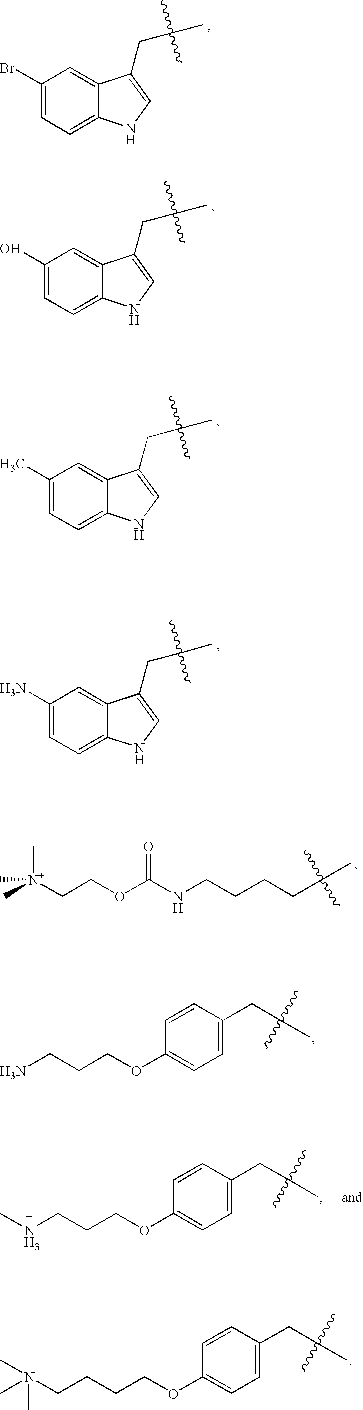

48. The screening method of claim 45 wherein said unnatural amino acids are represented by the Formula (II): 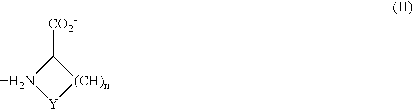
wherein: Y is CH₂, (CH)ₙ, N, O, or S, and n is 1 or 2.

49. The screening method of claim 45 wherein said unnatural amino acids are selected from the group consisting of: [chemical structure]
